(19) United States
(12) Patent Application Publication
(10) Pub. No.: US 2005/0244492 A1
(43) Pub. Date: Nov. 3, 2005

(54) Rapidly disintegrating tablets comprising titanium dioxide

(76) Inventors: Dev K. Mehra, Furlong, PA (US); Michael C. Withiam, Landenberg, PA (US); John M. Cornelius, Forest Hill, MD (US)

Correspondence Address: Carlos Nieves, Esq.;J. M. Huber Corporation, 333 Thornall Street, Edison, NJ, 08837-2220, US

(21) Appl. No.: 10/835,666

(22) Filed: Apr. 30, 2004

(51) Int. Cl. A61K 9/20; A61K 9/46; A61K 33/24

(52) U.S. Cl. 424/464; 424/617

(57) ABSTRACT

An orally-administered, rapidly disintegrating tablet is provided. The tablet comprises: a titanium dioxide; a super disintegrant; and a sugar alcohol. When immersed in water the tablet has a friability of less than about 2% and disintegrates in less than about 60 seconds.

### BACKGROUND OF THE INVENTION

Many consumer products, such as human and animal health and personal care products, are manufactured and packaged in solid, compacted form. The solid, compacted product form has several advantages over other product forms, such as relative ease of manufacture and durability in packaging and shipment and convenience in use and in storing for retailers and consumers alike. The compressed tablet form is particularly well-suited for oral care and hygiene.

However, in certain situations it would be beneficial if the tablet would disintegrate in the mouth so that tooth cleaning could be affected without the necessity of having access to a toothbrush or to water. For example, hikers, campers, boaters, or people traveling or eating in public places, could use an oral care tablet that rapidly disintegrates in the mouth providing a convenient and effective solid form delivery system for tooth cleaning and mouth freshening.

The administration of pharmaceuticals represents another situation where it is beneficial, if not extremely important, for the tablet to rapidly disintegrate in the mouth so that the active pharmaceutical is delivered to the blood stream of a patient much faster than a conventional tablet.. For example, children and advanced geriatric patients (those over 80 years old) often have difficulty swallowing pills, and a tablet that dissolves or rapidly disintegrates in the mouth would provide a convenient and effective solid form delivery system for such patients. Additionally, a tablet that dissolves, or disintegrates, in the mouth would be helpful for mentally disabled individuals who require treatment with pharmaceuticals, but refuse to swallow tablets.

Unfortunately, most tablets do not readily disintegrate in the mouth, but instead disintegrate in a slow and uneven fashion, for example when chewed. Given the forgoing there is a continuing need for solid form oral care preparations, including solid form orally-administered pharmaceutical preparations, that rapidly disintegrate in the mouth and that are not friable under packaging and shipping conditions.

### BRIEF SUMMARY OF THE INVENTION

The present invention includes an orally-administered, rapidly disintegrating tablet comprising (a) about 10% to about 80% titanium dioxide,(b) about 20% to about 80% of a sugar alcohol and (c) about 1% to about 30% of a super disintegrant.

### DETAILED DESCRIPTION OF THE INVENTION

All parts, percentages and ratios used herein are expressed by weight unless otherwise specified.

All publications, patent applications and issued patents mentioned herein are hereby incorporated in their entirety by reference.

The present invention relates to solid-form, orally-administered, rapidly disintegrating pharmaceutical products and personal care products that are oral care products in solid or semi-solid form such as dentifrices, toothpastes, and breath-fresheners; these personal care products may include titanium dioxides.

The solid-form, orally-administered, rapidly disintegrating pharmaceutical products and the oral care products of the present invention typically contain from about 10% to about 80% titanium dioxide, preferably from about 15% to about 50%, about 20% to 80% sugar alcohol, preferably about 20% to about 70%, and about 1% to about 30% of a super disintegrant, preferably about 3% to about 15%, more preferably about 3% to 5%.

Titanium dioxide provides dual finctionality to the rapidly disintegrating, solid-form, orally-administered pharmaceutical products and the oral care tablets. When included in orally-administered solid pharmaceutical products, such as a tablet, the tablet readily disintegrates in the mouth, and thus eliminates the need for swallowing the tablet in order to release the active pharmaceutical ingredient. Titanium dioxide is a water insoluble substance, which in the presence of a super disintegrant enables very rapid tablet disintegration when the tablet contacts water. Additionally, when incorporated into an oral care tablet, titanium dioxide serves as a dental abrasive providing tooth cleaning and polishing. Titanium dioxide may be rutile or anatase forms, which are often derived from ilmenite or leuxocene ores. A suitable source of titanium dioxide is the Tronox® titanium dioxide available from Kerr-McGee Pigments Oklahoma City, Okla.

The sugar alcohol provides multiple functions to the rapidly disintegrating oral care tablet. The sugar alcohol provides good aesthetic properties to the dissolved oral care tablet such as taste (sweetness and coolness due to its endothermal heat of solution) and “mouth texture” or body; aids in rapid tablet disintegration; and serves as a tablet filler. Suitable sugar alcohols are those given in The Encyclopedia of Chemical Technology, Vol. 23, 4^{th} Edition, Mary Howe-Grant, editor, John Wiley & Sons, New York, N.Y. (1997) pages 93-113, which is incorporated herein by reference, and include erythritol, xylitol, sorbitol, maltitol, mannitol, lactitol, and the like, used singly and in combinations, with mannitol and sorbitol preferred.

The super disintegrant facilitates the break-up of a tablet when it is placed in an aqueous environment, such as the mouth. Super disintegrants in contact with water swell, wick-in water or otherwise provide a disruptive force to a tablet causing it to break apart. Suitable super disintegrants include one or more of sodium starch glycolate, available as e.g. Explotab and Explosol; croscarmellose sodium (cross-linked sodium carboxymethyl cellulose) available as e.g. Ac-Di-Sol® and Nymcel® ZSX; and cross-linked polyvinylpyrolidone available as e.g. Polyplasdone XL.

In addition to the aforementioned ingredients, the oral care products of the present invention may also include several other ingredients such as additional disintegration aids, organoleptic enhancers, additional abrasives, thickening agents, (also sometimes known as thickeners, binders, gums, or stabilizing agents), therapeutic agents, and preservatives.

These solid formed oral care preparations may also include one or more disintegration aids, in addition to the super disintegrant. Suitable disintegration aids include natural, modified or pregelatinized starch; natural or chemically-modified cellulose; microcrystalline cellulose; gum, especially agar gum, and guar gum; alginic acid or salts thereof; acetates and citrates; sugars (especially sucrose, amylose, dextrose and lactose); aluminum oxide; synthetic polymers such as methacrylic acid-divinylbenzene copolymer, as well as effervescent disintegrating systems. Typical levels of disintegration aids in the inventive oral care preparations are from about 0.5% to about 15 % of the formulation, preferably from about 1% to about 5%.

The inventive oral care compositions may also contain one or more organoleptic enhancing agents. Organoleptic enhancing agents include humectants, sweeteners, surfactants, flavorants, colorants and effervescing agents.

Humectants serve to add body or “mouth texture” to a dentifrice. In addition to the previously mentioned sugar alcohols, suitable humectants include glycerin, polyethylene glycol (at a variety of different molecular weights), propylene glycol, and hydrogenated starch hydrolyzates, as well as mixtures of these compounds.

Sweeteners may be added to the dentifrice composition to impart a pleasing taste to the product. Suitable sweeteners include saccharin (as sodium, potassium or calcium saccharin), cyclamate (as a sodium, potassium or calcium salt), aspartame, acesulfane-K, thaumatin, neohisperidin dihydrochalcone, ammoniated glycyrrhizin, dextrose, maltodextrin, sucralose, fructose, levulose, sucrose, mannose, and glucose. Typical levels of sweeteners are from about 0% to about 5% of a dentifrice composition.

Surfactants are used in the compositions of the present invention to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants such as sodium lauryl sulfate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine and the like. Sodium lauryl sulfate is a preferred surfactant. The surfactant is typically present in the oral care compositions of the present invention in an amount of about 0.1 to about 15% by weight, preferably about 0.3% to about 5% by weight, such as from about 0.3% to about 2%, by weight.

Flavoring agents optionally can be added to dentifrice compositions. Suitable flavoring agents include, but are not limited to, oil of wintergreen, oil of peppermint, oil of spearmint, oil of sassafras, and oil of clove, cinnamon, anethole, menthol, thymol, eugenol, eucalyptol, lemon, orange and other such flavor compounds to add fruit notes, spice notes, etc. These flavoring agents consist chemically of mixtures of aldehydes, ketones, esters, phenols, acids, and aliphatic, aromatic and other alcohols.

Colorants may be added to improve the aesthetic appearance of the product. Suitable colorants are selected from colorants approved by appropriate regulatory bodies such as the FDA and those listed in the European Food and Pharmaceutical Directives and include pigments, such as TiO₂, and colors such as FD&C and D&C dyes.

The oral care product may also contain an effervescent agent to provide aesthetic properties to the tablet. Preferably effervescence is provided by reaction of a carbonate salt such as calcium carbonate, sodium carbonate, sodium bicarbonate, potassium carbonate or potassium bicarbonate with an acid such as citric acid, tartaric acid or malic acid.

In addition to titanium dioxide, the oral care table may contain additional abrasives. Suitable abrasives include precipitated and ground calcium carbonate, precipitated silica, such as Zeodent® silicas available from J. M. Huber Corporation, silica gel, calcium metasilicate, aluminum silicate,, alumina, calcined alumina, bentonite, particulate thermosetting resins and other suitable abrasive materials known to a person of ordinary skill in the art. The abrasive may be used alone or in combination with other abrasives. Typical levels of abrasives in the inventive dentifrice formulation are from about 2% to about 60%, preferably from about 2% to about 10%.

Thickening agents are useful in the oral care products of the present invention to provide a gelatinous structure that stabilizes the dentifrice against phase separation and provides an aesthetically pleasing texture when the composition disintegrates in the mouth. Suitable thickening agents include silica thickeners such as J. M. Huber Corporation Zeodent® precipitated silica products and silica gels available from Davison Chemical Division of W. R. Grace Corporation, Baltimore, Md. natural and synthetic clays such as hectorite clays; lithium magnesium silicate (laponite); and magnesium aluminum silicate (Veegum); starch; glycerite of starch; as well as mixtures of these compounds. Typical levels of thickening agents are from about 0% to about 15% of an oral care composition.

Therapeutic agents are optionally used in the compositions of the present invention to provide for the prevention and treatment of dental caries, periodontal disease and temperature sensitivity. Examples of therapeutic agents, without intending to be limiting, are fluoride sources, such as sodium fluoride, sodium monofluorophosphate, stannous fluoride, potassium fluoride, sodium fluorosilicate, ammonium fluorosilicate and the like; condensed phosphates such as tripolyphosphates, hexametaphosphates, trimetaphosphates and pyrophosphates; antimicrobial agents such as triclosan, bisguanides, such as alexidine, chlorhexidine and chlorhexidine gluconate; enzymes such as papain, bromelain, glucoamylase, amylase, dextranase, mutanase, lipases, pectinase, tannase, and proteases; quartemary ammonium compounds, such as benzalkonium chloride (BZK), benzethonium chloride (BZT), cetylpyridinium chloride (CPC), and domiphen bromide; metal salts, such as zinc citrate, zinc chloride, and stannous fluoride; sanguinaria extract and sanguinarine; volatile oils, such as eucalyptol, menthol, thymol, and methyl salicylate; amine fluorides; peroxides and the like. Therapeutic agents may be used in dentifrice formulations singly or in combination at a therapeutically safe and effective level.

Preservatives may be also be optionally added to the compositions of the present invention to prevent bacterial growth. Suitable preservatives approved for use in oral compositions such as methylparaben, propylparaben and sodium benzoate may be added in safe and effective amounts.

The oral care products may additionally contain other optional ingredients typically used in tablet making such as glidants to provide even flow to the granulation to be tabletted, e.g. amorphous silica such as Zeopharm® 80 (J. M. Huber Corporation, Edison, N.J.) and Cab-O-Sil® M5 (Cabot Corporation, Billerica, Mass.); die release aids, also known as lubricants, such as magnesium stearate (available as HYQUAL® NF from Mallinckrodt, Inc., St. Louis, Mo.) to enable tablets to be released from within the tablet machine die, anti-adherents, such as stearic acid, to facilitate separation of tablets from punch faces; and fillers such as microcrystalline cellulose, such as Avicel 101 (FMC Biopolymers, Philadelphia, Pa.) and Omnicel 102 (Functional Foods, Englishtown, N.J.).

All tablet formulation ingredients, except the lubricant, are weighed together and mixed. Thereafter, the lubricant is geometrically diluted with the just prepared tablet mixture and then added back to the mixture. This step is typically necessary to homogeneously incorporate the hydrophobic lubricant into the tablet mixture.

The tablets are then manufactured by using a tableting compacting process. A standard single stroke or a rotary press may be used. The tablets prepared according to this invention may be of any geometrical shape, such as round, square, triangular or caplet-shaped, and of any size suitable for human or animal use.

With regard to the rapidly disintegrating, solid-form, orally-administered pharmaceutical products of the present invention, these products, which are typically in tablet form, contain titanium dioxide, a sugar alcohol, a super disintegrant, and one or more pharmaceutically active ingredients. These pharmaceutical products may also contain the aforementioned tablet lubricants, glidants, one or more organoleptic agents and additional disintegration aids. Suitable pharmaceutically active ingredients include nourishing and health-promoting agents, antipyretic, analgesic, anti-inflammatory agents, antipsychotic drugs, PDE-5 inhibitors, antianxiety drugs, antidepressants, hypnotic-sedatives, spasmolytics, central nervous system affecting drugs, cerebral metabolism ameliolators, antiepileptics, sympathomimetic agents, gastrointestinal function conditioning agents, antacids, antiulcer agents, antitussive-expectorants, antiemetics, respiratory stimulants, bronchodilators, antiallergic agents, dental buccal drugs, antihistamines, cardiotonics, antiarrhythmic agents, diuretics, hypotensive agents, vasoconstrictors, coronary vasodilators, peripheral vasodilators, antihyperlipidemic agents, cholagogues, antibiotics, chemotherapeutic agents, antidiabetic agents, drugs for osteoporosis, skeletal muscle relaxants, antidinics, hormones, alkaloid narcotics, sulfa drugs, antipodagrics, anticoagulants, anti-malignant tumor agents, agents for Alzheimer's disease, etc. The titanium dioxide may also be included in veterinary pharmaceutical preparations.

The invention will now be described in more detail with respect to the following, specific, non-limiting examples.

### Tablet Preparation

Tablets were prepared by weighing all formulation ingredients together, except the lubricant magnesium stearate, on a weighing pan. Typically, a tablet formulation was 300 g to 500 g total weight, in order to prepare multiple tablets for testing. The combined ingredients were passed through a 20 mesh (850 μm) sieve to remove any lumps and then bag blended, by gentle inversion in a plastic bag for about 30 seconds of the formulation ingredients previously weighed. The resulting mixture was transferred to a PK-V blender (twin shell dry blender model 014-215-0053, available from Patterson Kelly, East Stroudsburg, Pa.) and mixed for 10 minutes. The magnesium stearate lubricant was then geometrically diluted with the mixture and then added back to the PK blender and all ingredients mixed together for an additional 5 minutes.

Tablets were formed from the resulting formulation on a 8-station Piccola rotary tablet press available from Riva S. A., Argentina, fitted with 10 mm standard concave die punches compacting over a range of compression forces. Tablet weight was set at 400 mg by adjusting the tablet press.

### Tablet Test Methods

All tablets were prepared 24 hours before testing hardness, disintegration time and friability.

Tablet hardness (H) expressed in kP, for each formulation, was measured on 5 tablets utilizing a Erweka TBH30 instrument (Milford, Conn.) and the result reported was an average of 5 measurements.

Tablet disintegration time was determined, according to the USP test for uncoated tablets by placing 6 tablets (each in a separate tube) in an Erweka ZT72 disintegrator (Milford, Conn.). The tablets were repeatedly immersed in 37° C. deionized water at a rate of 30 strokes/min. until the tablets disintegrated, as detected and recorded by the instrument. The reported result was an average of the 6 measurements.

Tablet friability was determined by placing 10 tablets in a Distek, Inc. Friabilator DF-3 (North Brunswick, N.J.) set for 100 revolutions. The % friability is calculated from the amount of tablet weight lost (friable) by weighing the tablets before and after rotation.

### EXAMPLES 1-4

In theses examples, tablet formulations were made with titanium dioxide (TiO₂), a super disintegrant, a sugar alcohol and other ingredients typically found in oral care formulations and in pharmaceutical tablet formulations. Formulations 1-3 represent placebo pharmaceutical tablet formulations. An active pharmaceutical ingredient could be substituted for a portion of the microcrystalline cellulose, mannitol and titanium dioxide, depending on the dosage desired. Formulations 3 and 4 are typical oral care tablet formulations containing ingredients typically found in oral care formulations, such as a surfactant, additional abrasive, an enzyme and sodium fluoride. These formulations were prepared according to the procedure described above with the amounts of ingredients identified in Table 1.

TABLE 1
Tablet Formulations

| | | Formulation No. | | | |
|---|---|---|---|---|---|
| | Source | 1 | 2 | 3 | 4 |
| TiO₂, % | Kerr-McGee | 40 | 25 | 10 | 20 |
| Tronox | Pigments | | | | |
| | Oklahoma City, OK | | | | |
| Mannitol, % | Roquette Freres | 38.25 | 53.25 | 63.25 | 0 |
| Pearlitol 200SD | Lestrem, France | | | | |
| Sorbitol, % | Sigma Chemicals | 0 | 0 | 0 | 8 |
| D-Sorbitol, | | | | | |
| min 98% | | | | | |
| MCC, % | Functional Foods | 15 | 15 | 20 | 16.39 |
| Omnicel 102 | Englishtown, NJ | | | | |
| Crospovidone, % | ISP Technologies, | 0 | 0 | 5 | 14 |
| Polyplasdone XL | Inc. Wayne, NJ | | | | |
| ExploTab, % | Penwest | 2.5 | 5 | 0 | 0 |
| Sodium starch | Pharmaceuticals | | | | |
| glycolate | Patterson., NY | | | | |
| Nymcel ZSK | Noviant | 2.5 | 0 | 0 | 0 |
| Croscarmellose | The Netherlands | | | | |
| sodium | | | | | |
| Sodium Lauryl | | 0 | 0 | 1 | 1 |
| Sulfate, % | | | | | |
| Cab-O-Sil M-5, % | Cabot Corporation | 1 | 1 | 1 | 1 |
| Silica glidant | Billerica, MA | | | | |
| Sodium fluoride, % | Sigma Chemicals | 0 | 0 | 0 | 0.01 |
| Papain, % | National Enzyme | 0 | 0 | 0 | 0.1 |
| | Forsyth, MO | | | | |
| Aspartame, % | Ajinomoto Co. | 0 | 0 | 0 | 3 |
| | Japan | | | | |
| Flavor, % | Invetech | 0 | 0 | 0 | 3 |
| Citrus blend | Tustin, CA | | | | |
| Sodium bicarbonate, | Arm & Hammer | 0 | 0 | 0 | 20 |
| Citric Acid, % | | 0 | 0 | 0 | 10 |
| Zeodent 9175, % | J. M. Huber Corp. | 0 | 0 | 0 | 3 |
| silica abrasive | Edison, NJ | | | | |
| Magnesium Stearate, | Malinckrodt | 0.75 | 0.75 | 0.75 | 0.5 |
| % | St. Louis, MO | | | | |

Tablets weighing 400 mg each were prepared according to the procedure described above. Each formulation was compressed into tablets at different compression forces for each respective formulation. The tablet hardness (H), disintegration time (DT) and Friability were determined according to the procedures described above for tablets pressed at different compression forces with the results summarized in Table 2 below.

TABLE 2
Tablet Properties

| Formulation | | | |
|---|---|---|---|
| No. | H (kP) | DT (sec) | % Friability |
| 1 | 2.75 | 24 | 1.45 |
| 1 | 4.14 | 16 | 1.45 |
| 2 | 3.28 | 28 | 0.60 |
| 2 | 7.03 | 23 | 0.35 |
| 2 | 7.34 | 33 | 0.2 |
| 3 | 3.18 | 8 | 0.785 |
| 3 | 7.73 | 12 | 0.278 |
| 3 | 11.31 | 15 | 0.176 |
| 4 | 2.82 | 41 | 0.71 |

It seen from the data above that all formulations provided tablets that could be compressed to an acceptable hardness providing friability of less than 2% and rapid disintegration greater than about 40 seconds. This small friability percentage reflects the tablets, despite their very fast disintegration times, are also strong and have excellent physical integrity. This means that they can remain intact during the periods of storage and transportation until being finally delivered to the consumer.

### COMPARATIVE EXAMPLE

For comparison, a tablet formulation containing the sugar alcohol mannitol and magnesium stearate lubricant, but no titanium dioxide and no super disintegrant, labeled Formulation C was prepared as described above. The formulation is given in Table 3 below.

TABLE 3
Comparative Example Tablet Formulation

| | Source | Formulation C |
|---|---|---|
| Mannitol, % | Roquette Freres | 99.25 |
| Pearlitol 200SD | Lestrem, France | |
| Magnesium | Malinckrodt | 0.75 |
| Stearate, % | | |

Tablets of Formulation C were made according to the procedure described above by compressing the tablets with different forces to provide tablets of differing hardness. These tablets were tested for hardness and disintegration time (DT) according to the methods previously described.

TABLE 4
Performance

| | Hardness (kP) | DT (s) |
|---|---|---|
| Formulation C | 3.5 | 42 |
| Formulation C | 10.0 | 165 |
| Formulation C | 13.4 | 145 |

It is seen from the above data that tablets without titanium dioxide and without a super disintegrant had longer disintegration times than tablets of comparable hardness made according to the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. An orally-administered, rapidly disintegrating tablet comprising:
a titanium dioxide;
a super disintegrant; and
a sugar alcohol;
wherein the tablet has a friability of less than about 2% and disintegrates when immersed in water in less than about 60 seconds.

2. The orally-administered tablet according to claim 1, wherein the tablet comprises about 10% to about 80 wt % of titanium dioxide.

3. The orally-administered tablet according to claim 1, wherein the super disintegrant is selected from one or more of sodium starch glycolate, croscarmellose sodium, and crospovidone.

4. The orally-administered tablet according to claim 1, wherein the tablet comprises about 1 wt % to about 30 wt % of the super disintegrant.

5. The orally-administered tablet according to claim 1, wherein the tablet comprises about 1 wt % to about 3 wt % of the super disintegrant

6. The orally-administered tablet according to claim 1, wherein the sugar alcohol is selected from one or more of sorbitol, mannitol, xylitol, erythritol, maltitol, and lactitol.

7. The orally-administered tablet according to claim 1, wherein the tablet comprises about 20 wt % to about 80 wt % of the sugar alcohol.

8. The orally-administered tablet according to claim 1, wherein the tablet friability is less than 1%.

9. The orally-administered tablet according to claim 1, wherein the tablet, when added to water at 37° C. disintegrants in less 40 seconds.

10. The orally-administered tablet according to claim 1, further comprising one or more ingredients selected from the group consisting of: organoleptic enhancing agents, preservatives, and disintegration aids.

11. The orally-administered tablet according to claim 10, wherein the organoleptic enhancing agent comprises one or more ingredients selected from the group consisting of humectants, sweeteners, flavorants, surfactants, colorants and effervescent agents.

12. The orally-administered tablet according to claim 1, wherein the tablet is a pharmaceutical tablet and further comprises a pharmaceutically active ingredient.

13. The orally-administered tablet according to claim 1, wherein the tablet is an oral care tablet and further comprises one or more ingredients selected from the group consisting of: abrasives, therapeutic agents, surfactants, and thickening agents.

14. An orally-administered, rapidly disintegrating tablet comprising:
about 10 wt % to about 80 wt % titanium dioxide;
about 1 wt % to about 15 wto/o super disintegrant;
about 20 wt % to about 80 wt % sugar alcohol; and
about 0.1 wt % to about 5 wt % surfactant.

15. The orally-administered tablet according to claim 14, wherein the tablet has a friability of less than about 2% and the tablet disintegrates when immersed in water in less than about 60 seconds.

16. The orally-administered tablet according to claim 14, further comprising a flavorant.
